(19) United States
(12) Patent Application Publication
(10) Pub. No.: US 2004/0167407 A1
(43) Pub. Date: Aug. 26, 2004

(54) Capacitive DC-to-DC converter with efficient use of flying capacitors and related method

(75) Inventor: Jonathan P. Roberts, Shoreview, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/366,219

(22) Filed: Feb. 13, 2003

(51) Int. Cl. A61B 5/02

(52) U.S. Cl. 600/485

(57) ABSTRACT

A DC-to-DC converter (120) is suitable for use in an implantable medical device (5) and includes N capacitors (160, 170) and a controller (140). N is a whole number greater than one. The controller (140) is coupled to each of the N capacitors (160,170) and to an output voltage terminal (142) and has N+1 phases. During a first phase of the N+1 phases the controller (140) couples selected ones of the N capacitors (160, 170) to the output voltage terminal (142). During successive N phases of the N+1 phases the controller (140) couples other selected ones of the N capacitors (160, 170) selectively between an input voltage terminal, a power supply voltage terminal, and the output voltage terminal (142). Voltages across each capacitor of the N capacitors (160, 170) and at the output voltage terminal (142) assume respective uniquely determined values during all of the N+1 phases.

### TECHNICAL FIELD

This invention relates generally to voltage converter circuits, and more particularly, to capacitive voltage converters.

### BACKGROUND OF THE INVENTION

In some electronic circuits it is necessary to convert a constant (DC) power supply voltage to a different value. A circuit which performs the conversion is referred to as a DC-to-DC converter. For example in an implantable medical device the operating power is supplied by a small battery. The battery voltage will vary over a wide range during its operating life. Thus it is necessary to convert the battery's voltage to one or more suitable voltages for use in the device.

It is important to convert the battery voltage to the lowest possible voltage to minimize the power consumed by digital circuits. In digital circuits power consumption is primarily caused by two mechanisms. First the amount of power consumed by switching logic states on the gate of a transistor is proportional to the gate capacitance times the square of the voltage change. A smaller voltage change due to reduced swing in logic states will reduce this component of power consumption. Second digital CMOS circuits draw current between power and ground through the transistors, known as crowbar current, when switching logic states. A lower power supply voltage reduces crowbar current. It is also important to operate digital circuitry at low voltage from a reliability standpoint. As device geometries decrease, breakdown voltages also decrease. Furthermore sub-threshold leakage increases as device geometries decrease.

Analog circuits need a constant supply voltage that does not vary as the battery voltage changes. A constant voltage eases the constraints on circuit design by decreasing the need for circuits with very high power supply rejection ratios. Analog circuits also frequently need a higher voltage than digital circuits to maintain voltage headroom in amplifiers.

It is also important to maximize the battery life of the battery-powered implantable medical device, because replacing the battery involves an invasive surgical procedure. Thus the DC-to-DC converter must be as power efficient as possible. Other battery-powered electronic devices including personal computers, cellular telephones, personal digital assistants, and the like also share the need to be power efficient because changing batteries is inconvenient to the user.

When the output voltage needs to be less than the input voltage, three types of DC-to-DC converters have been used. The first type of converter is a linear regulator. The linear regulator reduces the output voltage relative to the input voltage but maintains a constant current. The linear regulator approach is inefficient from a power standpoint and is not suitable for applications that require minimal power consumption, such as battery-powered implantable medical devices.

The second type of a DC-to-DC converter is a switching regulator. A switching regulator uses a coil inductor and can achieve efficiencies of approximately 85-90%. However the coil can saturate in high magnetic fields and it is susceptible to electromagnetic interference (EMI) and radio frequency (RF) noise. It also becomes a source of EMI to other adjacent devices.

The third type of DC-to-DC converter is a capacitor-based converter, also known as a charge pump converter. This type of converter can also achieve up to 90% power efficiency. A typical capacitor-based converter, such as one based on the TPS60500 available from Texas Instruments of Dallas, Tex., uses an integrated circuit with external “flying” capacitors and an external output capacitor. The TPS60500 switches two flying capacitors in a particular sequence during a charging mode and a discharging mode to operate in ⅓, ½, ⅔, and low dropout (LDO) modes. The TPS60500 automatically changes modes as the output voltage varies. So for example if a battery has an initial voltage of slightly above 4 volts and the desired output voltage is 2 volts, the TPS60500 initially selects the ½ mode, but when the output voltage drops below 2 volts due to battery discharge, the TPS60500 automatically switches to the ⅔ mode.

As the input voltage departs from the ideal ratio, however, additional regulation is required. The TPS60500 regulates the output voltage by using a feedback input signal and by modulating the amount of current driven into the capacitors. Alternatively a linear regulator could be connected to the output of a capacitive DC-to-DC converter to achieve a more precise output voltage. As the desired output voltage departs from the supported ratios and requires additional regulation, power efficiency goes down This reduction in efficiency is highly undesirable. What is needed then is a capacitor-based converter that provides high power efficiency across a wide range of input voltages using a small number of flying capacitors. Such a converter is provided by the present invention, whose features and inventive aspects will become more clearly understood from the following detailed description taken in conjunction with the accompanying drawings.

### SUMMARY OF THE INVENTION

Accordingly the present invention provides, in one form, a DC-to-DC converter suitable for use in an implantable medical device including N capacitors and a controller. N is a whole number greater than one. The controller is coupled to each of the N capacitors and to an output voltage terminal and has N+1 phases. During a first phase of the N+1 phases the controller couples selected ones of the N capacitors to the output voltage terminal. During successive N phases of the N+1 phases the controller couples other selected ones of the N capacitors selectively between an input voltage terminal, a power supply voltage terminal, and the output terminal. Voltages across each capacitor of the N capacitors and at the output voltage terminal thereby assume respective uniquely determined values during all of the N+1 phases.

In another form the present invention provides an implantable medical device comprising a sensor, a monitor circuit, a power source, and a DC-to-DC converter. The sensor is adapted to be coupled to living tissue. The monitor circuit is coupled to the sensor, processes inputs received from the sensor, and is powered with an output power supply voltage. The DC-to-DC converter has an input coupled to the power source and an output coupled to the monitor circuit to provide the output power supply voltage thereto. The DC-to-DC converter includes N capacitors and a controller. N is a whole number greater than one. The controller is coupled to each of the N capacitors and to an output voltage terminal and has N+1 phases. During a first phase of the N+1 phases the controller couples selected ones of the N capacitors to the output voltage terminal. During successive N phases of the N+1 phases the controller couples other selected ones of the N capacitors selectively between an input voltage terminal, a power supply voltage terminal, and the output voltage terminal. Voltages across each capacitor of the N capacitors and at the output voltage terminal thereby assume respective uniquely determined values during all of the N+1 phases.

In yet another form the present invention provides a method for converting an input voltage to an output voltage. N capacitors are provided, wherein N is a whole number greater than one, and N+1 phases are generated. Selected ones of the N capacitors are coupled to an output voltage terminal during a first phase. Other selected ones of the N capacitors are coupled selectively between an input voltage terminal, the output voltage terminal, and a power supply voltage terminal during each remaining phase. Voltages across each capacitor of the N capacitors and at the output voltage terminal thereby assume respective uniquely determined values during all of the N+1 phases.

### BRIEF DESCRIPTION OF THE DRAWINGS

The following drawings are illustrative of particular embodiments of the invention and therefore do not limit the scope of the invention, but are presented to assist in providing a proper understanding. The drawings are not to scale and are intended for use in conjunction with the explanations in the following detailed description. The present invention will hereinafter be described in conjunction with the appended drawings, wherein like numerals denote like elements, and:

FIG. 1 illustrates a simplified block/schematic diagram of an implantable medical device according to the present invention;

FIG. 2 illustrates in partial block diagram and partial schematic form the DC-to-DC converter of FIG. 1;

FIGS. 3a-3c illustrate in schematic form the operation of the DC-to-DC converter of FIG. 2 when N=2 and M=3;

FIGS. 4a-4c illustrate in schematic form the operation of the DC-to-DC converter of FIG. 2 when it is used to generate a boosted output voltage; and

FIGS. 5a-5d illustrate in schematic form the operation of the DC-to-DC converter of FIG. 2 when N=3 and M=7.

### DETAILED DESCRIPTION OF EXEMPLARY EMBODIMENTS

The following description is exemplary in nature and is not intended to limit the scope, applicability, or configuration of the invention in any way. Rather the following description provides a convenient illustration for implementing exemplary embodiments of the invention. Various changes to the described embodiments may be made in the function and arrangements of the elements described herein without departing from the scope of the invention.

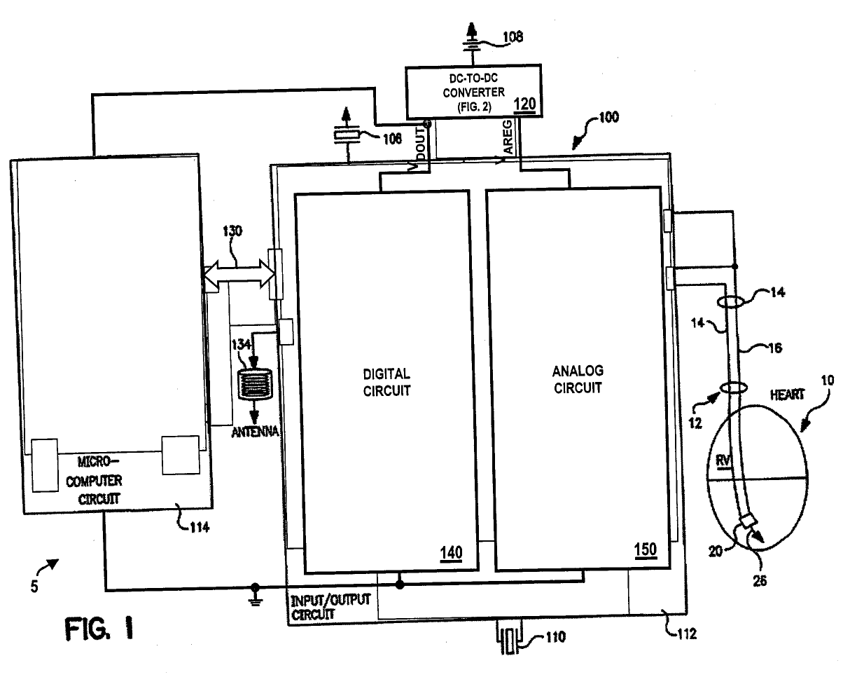

FIG. 1 illustrates a simplified block/schematic diagram of an implantable medical device 5 according to the present invention. In device 5 a capacitive pressure sensing lead 12 is designed to chronically transduce blood pressure from the right ventricle of a heart 10. Lead 12 is primarily employed with an implantable, battery powered monitor 100 which employs a microprocessor based demodulation, data storage, and telemetry system for sampling and storing blood pressure data at programmed intervals and telemetering out the accumulated data to an external programmer/transceiver on receipt of a programmed-in command, in the manner of current, conventional multi-programmable pacemaker technology. Lead 12 is intended to be implanted transvenously into the right heart chambers in the same manner as a conventional pacing lead, except that the distal end, including the pressure sensor module, may be advanced out of the right ventricle into the pulmonary artery to monitor blood pressure in that location. The monitor is intended to be implanted subcutaneously in the same manner that pacemakers are implanted.

Device 5 includes generally pressure sensing lead 12 and a monitor 100. Lead 12 has first and second lead conductors 14 and 16 extending from a proximal connector end to a pressure sensor module 20 disposed near a distal tine assembly 26. Pressure sensor module 20 includes a variable pickoff capacitor, a fixed reference capacitor, and a signal modulating circuit as further described in U.S. Pat. No. 6,171,252 which is herein incorporated by reference. The proximal connector assembly is formed as a conventional bipolar, in-line pacing lead connector and is coupled to the monitor connector (not shown) which is formed as a conventional bipolar in-line pacemaker pulse generator connector block assembly. Tine assembly 26 comprises soft pliant tines adapted to catch in heart tissue to stabilize lead 12 in a manner well known in the pacing art.

Monitor 100 includes an optional activity sensor 106, a battery 108, a crystal 110, an input/output circuit 112, a microcomputer circuit 114, a DC-to-DC converter 120, a data communication bus 130, and a telemetry antenna 134. Input/output circuit 112 includes circuitry designed to interface between microcomputer circuit 114 and the various components in device 5. These circuits include a crystal oscillator circuit connected to crystal 110, a power-on-reset (POR) circuit, a bias voltage generation circuit, an analog-to-digital converter (ADC) circuit, a radio frequency (RF) transmitter/receiver circuit connected to telemetry antenna 134, an optional activity circuit connected to optional activity sensor 106, a pressure signal demodulator connected to lead 12, and a digital controller/timer circuit connected to microcomputer circuit 114 over data communication bus 130. Further details of the operation of these components are contained in U.S. Pat. No. 6,151,272.

The circuits within input/output circuit 112 are separated into a digital circuit 140 and an analog circuit 150. DC-to-DC converter 120 is connected to battery 108 and provides separate power supply voltages to microcomputer circuit 114 and digital circuit 140 on the one hand, and to analog circuit 150 on the other. DC-to-DC converter 120 provides a digital power supply voltage labeled “V_{DOUT}” such that digital circuit 140 and microcomputer circuit 114 receive their operating power between V_{DOUT} and ground. DC-to-DC converter 120 provides an analog power supply voltage signal labeled “V_{AREG}” such that analog circuit 150 receives its operating power between V_{AREG} and ground. DC-to-DC converter 120 advantageously provides these two power supply voltages at different levels to satisfy the different requirements of digital circuitry in microcomputer circuit 114 and digital circuit 140, and in analog circuitry in analog circuit 150 while minimizing power consumption. V_{DOUT} is provided at a value just above the minimum voltage required for operation of the digital circuitry to minimize power consumption. V_{AREG} is provided at a higher voltage that is more suitable for operation of analog circuitry and is carefully regulated to a constant voltage. In the illustrated embodiment battery 108 provides an output voltage of about 3.5 volts early in its life, V_{DOUT} is preferably about 1.2 volts and V_{AREG} is preferably about 1.8 volts. As the voltage of battery 108 decreases over its operating life, DC-to-DC converter 120 automatically changes its operating characteristics so that V_{DOUT} and V_{AREG} are generated in the most power efficient manner, as will be described more fully below.

DC-to-DC converter 120 is ideally suited for use in implantable medical device 5 because it is very power efficient and thus will maximize the useful life of the device. DC-to-DC converter 120 can be used in various types of implantable medical devices including hemodynamic monitors, cardiac pacemakers, defibrillators, and the like. Furthermore it is suitable for powering other types of devices that require extremely low power consumption such as personal computers, cellular telephones, personal digital assistants (PDAs), and the like.

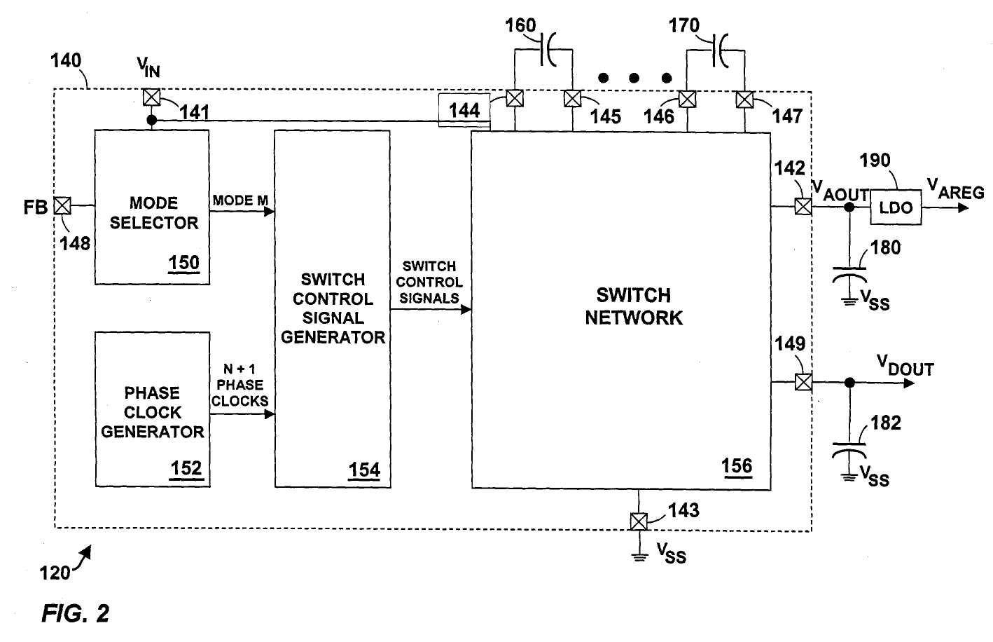

FIG. 2 illustrates in partial block diagram and partial schematic form DC-to-DC converter 120 of FIG. 1. Converter 120 includes generally a controller 140, capacitors 160, 170, 180, and 182, and a voltage regulator labeled “LDO” 190. Controller 140 is conveniently implemented as an integrated circuit having terminals 141-149. Terminal 141 is an input terminal for receiving an input power supply voltage from battery 108 labeled “V_{IN}”. Terminal 142 is an output terminal for providing a power supply voltage for analog circuitry labeled “V_{AOUT}”. Terminal 143 is a ground terminal connected to a ground p power supply voltage labeled “V_{SS}”. Terminals 144 and 145 are connected to the positive and negative terminals, respectively, of a first flying capacitor 160. Terminals 146 and 147 are adapted to be coupled to the positive and negative terminals, respectively, of an N^{th} flying capacitor 170. Terminal 148 is an input terminal for receiving a feedback voltage labeled “FB”. Terminal 149 is an output terminal for providing a second power supply voltage for digital circuitry, namely V_{DOUT}. Note that capacitors 160 and 170 are illustrated as having positive and negative terminals to distinguish the terminals but they need not be polarized capacitors. Capacitor 180 has a first terminal connected to output terminal 142 and a second terminal connected to V_{SS}. Capacitor 182 has a first terminal connected to output terminal 149 and a second terminal connected to V_{SS}. Voltage regulator 190 has an input terminal connected to terminal 142, and an output terminal for providing V_{AREG}.

Controller 140 includes a mode selector 150, a phase clock generator 152, a switch control signal generator 154, and a switch network 156. Mode selector 150 is coupled to terminals 141 and 148 and provides an output signal labeled “MODE M” that selects the M^{th} one of 2^{N} possible modes. Phase clock generator 152 generates a clock signal having at least N+1 phases and provides corresponding phase clock signals at an output labeled “N+1 PHASE CLOCKS”. Switch control signal generator 154 provides outputs labeled “SWITCH CONTROL SIGNALS” which connect the terminals of each of the N capacitors to various other terminals during their appropriate phases to implement the selected mode (MODE M).

Generally, converter 120 forms a DC-to-DC converter using N capacitors, where N is a whole number greater than one. Converter 120 switches the N capacitors selectively during N+1 phases to provide a selected one of 2^{N} output levels. Converter 120 provides V_{OUT} (one of V_{AOUT} and V_{DOUT}) at

$$( \frac{M}{{2}^{N}} )$$

times V_{IN}, where M=1 to 2^{N}. Thus if N=2, converter 120 uses two flying capacitors, has at least three phases, and is able to provide V_{OUT} at

$$( \frac{M}{4} )$$

times V_{IN}, where M=1 to 4, or at the ratios ¼, ½, ¾ and 1. Similarly if N=3, converter 120 uses three flying capacitors, has at least four phases, and is able to provide V_{OUT} at

$$( \frac{M}{8} )$$

times V_{IN}, where M=1 to 8 or at the ratios

$$\frac{1}{8} , 1 / 4 , \frac{3}{8} , 1 / 2. \frac{5}{8} , 3 / 4 , \frac{7}{8} ,$$

and 1.

More particularly mode selector 150 automatically determines the proper mode to provide the closest output voltage V_{AOUT} to the desired output voltage from the actual V_{IN} in response to the feedback voltage FB. In many battery-powered applications V_{IN} will vary over a wide range and mode selector 150 automatically changes the selected mode during operation to achieve optimum power efficiency. For example, the V_{IN} may nominally be 3.5 volts and the desired V_{AOUT} is 2 volts. Mode selector selects an initial setting of ⅝ mode (i.e., M=5). Voltage regulator 190 further regulates V_{AOUT} from 3.5*⅝=2.1875 volts down to 2 volts. When the battery drains such that regulator 190 can no longer keep V_{OUT} at 2 volts, mode selector 150 automatically changes the mode to ¾ mode (i.e., M=6).

FB is derived from V_{AREG} and in the illustrated embodiment is equal to V_{AREG}. Mode selector 150 internally generates 2^{N} voltages between V_{IN} and V_{SS} using a resistor string. The M^{th} voltage and adjacent voltages are compared to FB in order to determine whether to change the mode. In one alternative embodiment all 2^{N} voltages could be compared with FB in a flash comparator arrangement. In another alternative embodiment V_{AREG} could be divided by a resistor string to generate FB such that when V_{AREG} is equal to the desired value, FB equals a bandgap reference voltage. In this alternative embodiment mode selector 150 would include a bandgap voltage reference circuit to provide the bandgap reference voltage. Capacitors 180 and 182 serve as smoothing capacitors to smooth their respective power supply output voltages.

In accordance with the present invention converter 120 is able to simultaneously generate a separate output voltage V_{DOUT} using a different ratio than it used to generate V_{AOUT}. For the example of V_{IN}=3.5 volts, M would be selected to be 3, and V_{DOUT}=⅜ times 3.5=1.3125 volts. Since the operation of digital circuitry is not very sensitive to exact power supply voltages, providing V_{DOUT} above the desired voltage of 1.2 volts is acceptable and a separate linear regulator is not required.

Controller 140 switches the N capacitors during the N+1 clock phases so that a voltage across each capacitor and at the output terminal assume respective uniquely determined values during all of the clock phases. Expressed algebraically, there are N+1 variables and N+1 simultaneous voltage equations, yielding one solution. Since the capacitors assume their respective uniquely determined value during all N+1 clock phases, the only steady-state current is the current drawn by the load and thus the overall power efficiency is very high.

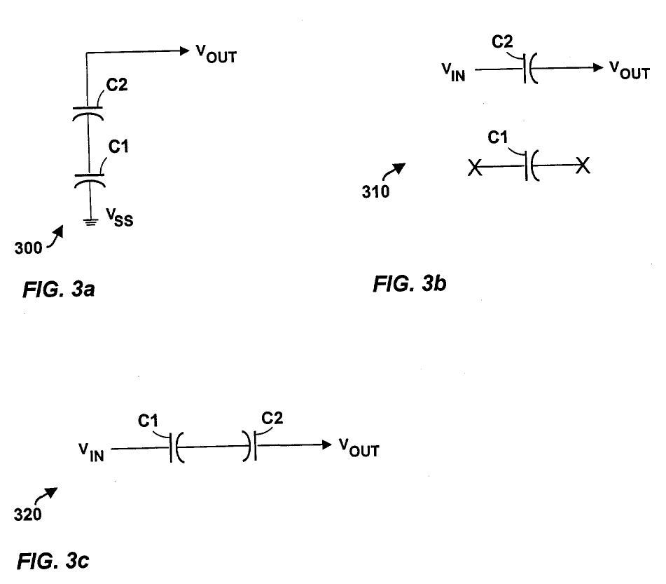

To demonstrate the operation three concrete examples will be shown. First, FIGS. 3a-3c illustrate in schematic form the operation of DC-to-DC converter 120 of FIG. 2 when N=2 and M=3 (i.e., ¾ mode). Switch network 156 makes the appropriate connections during each of the three required phases using switching elements based on the SWITCH CONTROL SIGNALS output by switch control signal generator 154. FIG. 3a illustrates a configuration 300 during a first phase in which the negative terminal of capacitor C1 (one of capacitors 160 and 170) is connected to V_{SS}, the positive terminal of C1 is connected to the negative terminal of a capacitor C2 (the other one of capacitors 160 and 170), and the positive terminal of capacitor C2 is connected to V_{OUT}, where V_{OUT} can be either V_{AOUT} or V_{DOUT}. FIG. 3b illustrates a configuration 310 during a second phase in which capacitor C1 is left unconnected, the negative terminal of capacitor C2 is connected to V_{OUT}, and the positive terminal of capacitor C2 is connected to V_{IN}. FIG. 3c illustrates a configuration 320 during a third phase in which the negative terminal of capacitor C1 is connected to the negative terminal of capacitor C2, the positive terminal of capacitor C1 is connected to V_{IN}, and the positive terminal of capacitor C2 is connected to V_{OUT}. When capacitors C1 and C2 are configured and switched in the sequence illustrated in FIGS. 3a-3c, V_{OUT} will equal ¾ V_{IN} because the only solution to the three simultaneous voltage equations is for V(C1)=¼ V_{IN}, V(C2)=½ V_{IN}, and V_{OUT}=¼ V_{IN}. Note that in an actual embodiment it may be more convenient to generate a four-phase clock and as long as the connections in the fourth phase remain the same as in one of the other phases, V_{OUT} will still be generated at the desired value.

The connections in the case of N=2 during each of the three phases for all values of M from 1 to 4 are illustrated in TABLE I below, in which NC means no connection, C1− is the negative terminal of capacitor C1, C1+ is the positive terminal of capacitor C1, and so on:

TABLE I

| | Phase | C1− | C1+ | C2− | C2+ |
|---|---|---|---|---|---|
| M = 1 | 1 | V_{OUT} | C2− | C1+ | V_{IN} |
| | 2 | V_{SS} | V_{OUT} | NC | NC |
| | 3 | V_{OUT} | C2+ | V_{SS} | C1+ |
| M = 2 | 1 | V_{OUT} | V_{IN} | V_{OUT} | V_{IN} |
| | 2 | V_{SS} | V_{OUT} | V_{SS} | V_{OUT} |
| | 3 | V_{SS} | V_{OUT} | V_{SS} | V_{OUT} |
| M = 3 | 1 | V_{OUT} | V_{IN} | NC | NC |
| | 2 | C2− | V_{OUT} | C1− | V_{IN} |
| | 3 | C2+ | V_{OUT} | V_{SS} | C1− |
| M = 4 | 1 | NC | NC | NC | NC |
| | 2 | NC | NC | NC | NC |
| | 3 | NC | NC | NC | NC |

Note that for the case of M=4, V_{IN} is connected directly to V_{OUT} during all phases. Also for the case of M=2 both capacitors had the same connections during all phases to provide a lower impedance at the output, but would be connected differently to support simultaneous output mode, described more fully below.

By using N+1 phases converter 120 can generate uniform output voltages to increase the granularity of available conversion ratios. Since the departure from the closest supported ratio can be made small, power efficiency will increase. Also converter 120 can support additional known modes besides the M/2^{N} modes, such as ⅓ and ⅔ modes.

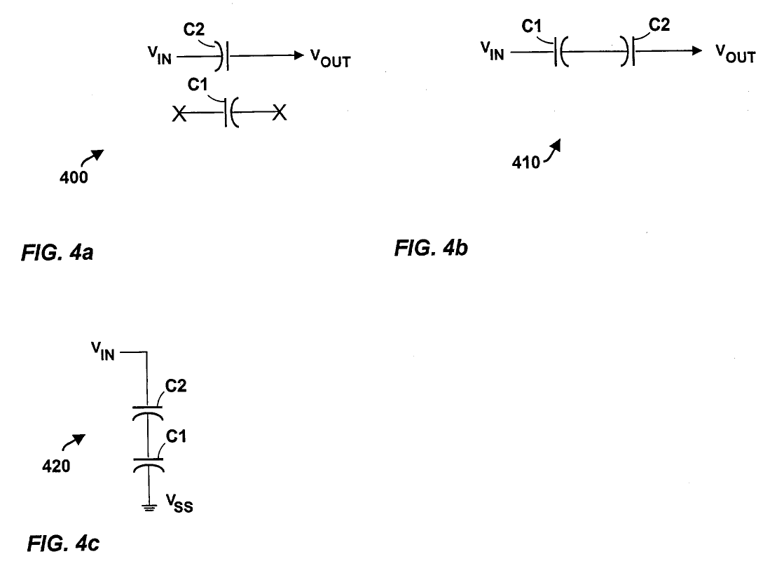

Another advantage of this architecture is that converter 120 can supporting a voltage boosting mode as well. One such configuration is shown in FIG. 4a-4c, which illustrate in schematic form the operation of DC-to-DC converter 120 of FIG. 2 when it is used to generate a boosted output voltage. FIG. 4a illustrates a configuration 400 during a first phase in which capacitor C1 is unconnected, the negative terminal of capacitor C2 is connected to V_{IN}, and the positive terminal of a capacitor C2 is connected to V_{OUT}. FIG. 4b illustrates a configuration 410 during a second phase in which the negative terminal of capacitor C1 is connected to the negative treminal of capacitor C2, the positive terminal of capacitor C1 is connected to V_{IN} and the positive terminal of capacitor C2 is connected to V_{OUT}. FIG. 4c illustrates a configuration 420 during a third phase in which the negative terminal of capacitor C1 is connected to V_{SS}, the positive terminal of capacitor C1 is connected to the negative terminal of capacitor C1, and the positive terminal of capacitor C2 is connected to V_{IN}. This switching sequence provides V_{OUT} at 4/3 times V_{IN}.

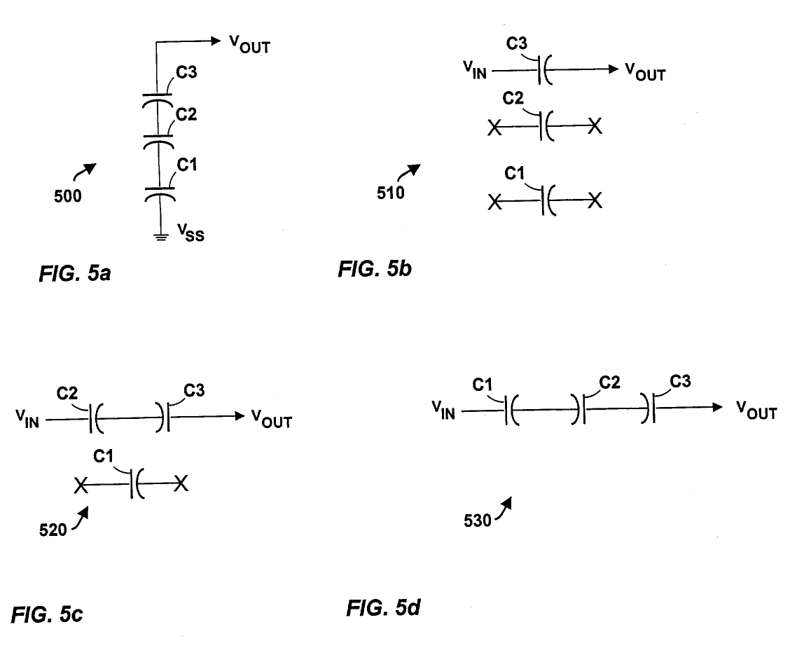

FIGS. 5a-5d illustrate in schematic form the operation of converter 120 of FIG. 2 when N=3 and M=7 (i.e. ⅞ mode). Thus FIGS. 5a-5d include a third capacitor C3 and four phases. FIG. 5a illustrates a configuration 500 during a first phase in which the negative terminal of C1 is connected to V_{SS}, the positive terminal of C1 is connected to the negative terminal of C2, the positive terminal of C2 is connected to the negative terminal of C3, and the positive terminal of C3 is connected to V_{OUT}. FIG. 5b illustrates a configuration 510 during a second phase in which C1 and C2 are not connected, i.e., floating or isolated, the negative terminal of C3 is connected to V_{OUT}, and the positive terminal of C3 is connected to V_{IN}. FIG. 5c illustrates a configuration 520 during a third phase in which C1 is not connected, the negative terminal of C2 is connected to the negative terminal of C3, the positive terminal of C2 is connected to V_{IN}, and the positive terminal of C3 is connected to V_{OUT}. FIG. 5d illustrates a configuration 530 during a fourth phase in which the negative terminal of C1 is connected to the negative terminal of C2, the positive terminal of C1 is connected to V_{IN}, the positive terminal of C2 is connected to the negative terminal of C3, and the positive terminal of C3 is connected to V_{OUT}.

The connections during each of the four phases for all values of M are illustrated in TABLE II below:

TABLE II

| | Phase | C1− | C1+ | C2− | C2+ | C3− | C3+ |
|---|---|---|---|---|---|---|---|
| M = 1 | 1 | V_{SS} | C2+ | C3+ | C1+ | V_{OUT} | C2− |
| | 2 | NC | NC | V_{SS} | C3+ | V_{OUT} | C2+ |
| | 3 | NC | NC | NC | NC | V_{SS} | V_{OUT} |
| | 4 | C2+ | V_{IN} | C3+ | C1− | V_{OUT} | C2− |
| M = 2 | 1 | V_{SS} | C2+ | V_{OUT} | C1+ | NC | NC |
| | 2 | NC | NC | V_{SS} | V_{OUT} | NC | NC |
| | 3 | C2+ | V_{IN} | V_{OUT} | C1− | NC | NC |
| | 4 | C2+ | V_{IN} | V_{OUT} | C1− | NC | NC |
| M = 3 | 1 | V_{SS} | C3+ | NC | NC | V_{OUT} | C1+ |
| | 2 | NC | NC | V_{SS} | C3− | C2+ | V_{OUT} |
| | 3 | V_{SS} | C2+ | C3− | C1− | C2− | V_{OUT} |
| | 4 | C3+ | V_{IN} | NC | NC | V_{OUT} | C1− |
| M = 4 | 1 | V_{SS} | V_{OUT} | NC | NC | NC | NC |
| | 2 | V_{SS} | V_{OUT} | NC | NC | NC | NC |
| | 3 | V_{OUT} | V_{IN} | NC | NC | NC | NC |
| | 4 | V_{OUT} | V_{IN} | NC | NC | NC | NC |
| M = 5 | 1 | V_{SS} | C2− | C1+ | C3+ | V_{OUT} | C2+ |
| | 2 | NC | NC | C3+ | V_{IN} | V_{OUT} | C2− |
| | 3 | C2+ | V_{IN} | C1− | C3+ | V_{OUT} | C2+ |
| | 4 | C3− | V_{IN} | NC | NC | C1− | V_{OUT} |
| M = 6 | 1 | V_{SS} | C2− | C1+ | V_{OUT} | NC | NC |
| | 2 | V_{SS} | C2− | C1+ | V_{OUT} | NC | NC |
| | 3 | NC | NC | V_{OUT} | V_{IN} | NC | NC |
| | 4 | C2− | V_{IN} | C1− | V_{OUT} | NC | NC |
| M = 7 | 1 | V_{SS} | C2− | C1+ | C3− | C2+ | V_{OUT} |
| | 2 | NC | NC | NC | NC | V_{OUT} | V_{IN} |
| | 3 | NC | NC | C3− | V_{IN} | C2− | V_{OUT} |
| | 4 | C2− | V_{IN} | C1− | C3− | C2+ | V_{OUT} |
| M = 8 | 1 | NC | NC | NC | NC | NC | NC |
| | 2 | NC | NC | NC | NC | NC | NC |
| | 3 | NC | NC | NC | NC | NC | NC |
| | 4 | NC | NC | NC | NC | NC | NC |

Note that for the case of M=8, V_{IN} is connected directly to V_{OUT} during all phases. Also for the case of M=4 all three capacitors could have the same connections during all phases to provide a lower impedance at the output, but would be connected differently to support simultaneous output mode, described more fully below.

To demonstrate how converter 120 causes the output voltage that is expected to occur at V_{OUT}, the case of M=7 and N=3 will be analyzed. From the four pump phases, voltage equations can be written:

V_{C1}+V_{C2}+V_{C3}=V_{OUT} [1]

V_{IN}−V_{C3}=V_{OUT} [2]

V_{IN}−V_{C2}+V_{C3}=V_{OUT} [3]

V_{IN}−V_{C1}+V_{C2}+V_{C3}=V_{OUT} [4]

Since V_{IN} is a known value, this yields four equations with four unknowns and can be solved algebraically. The following steps can be used to solve for all variables. First, Equation [1] can be used to substitute V_{C1}+V_{C2}+V_{C3} for V_{OUT} in Equations [2]-[4]:

V_{IN}−V_{C3}=V_{C1}+V_{C2}+V_{C3} [5]

V_{IN}−V_{C2}+V_{C3}=V_{C1}+V_{C2}+V_{C3} [6]

V_{IN}−V_{C1}+V_{C2}+V_{C3}=V_{C1}+V_{C2}+V_{C3} [7]

Next, rearrange Equations [5]-[8]:

V_{IN}=V_{C1}+V_{C2}+2V_{C3} [8]

V_{IN}=V_{C1}+2V_{C2} [9]

V_{IN}=2V_{C1} [10]

$${V}_{C1} = \frac{{V}_{IN}}{2} \qquad [ 11 ]$$

Now replace the result into the other equations:

$$\begin{matrix}{V}_{IN} = \frac{{V}_{IN}}{2} + {V}_{C2} + 2 {V}_{C3} & [ 12 ] \\ {V}_{IN} = \frac{{V}_{IN}}{2} + 2 {V}_{C2} & [ 13 ]\end{matrix}$$

Giving:

$$\begin{matrix}\frac{{V}_{IN}}{2} = {V}_{C2} + 2 {V}_{C3} & [ 14 ] \\ \frac{{V}_{IN}}{4} = {V}_{C2} & [ 15 ]\end{matrix}$$

Substituting

$${V}_{C2} = \frac{{V}_{IN}}{4}$$

into the remaining equation yields

$${V}_{C3} - \frac{{V}_{IN}}{8} .$$

Now we can substitute these results back into the original equation to find V_{OUT}:

$${V}_{OUT} = {V}_{C1} + {V}_{C2} + 2 {V}_{C3} = \frac{{V}_{IN}}{2} + \frac{{V}_{IN}}{4} + \frac{{V}_{IN}}{8} = \frac{7}{8} {V}_{IN} \qquad [ 16 ]$$

Another advantage of this architecture is that it allows multiple, simultaneous output voltage generation. Since the flying capacitors do not change their voltages between phases, the same flying capacitors can be shared between multiple outputs. Such a configuration is equivalent to a 2(N+1) phase pump with two outputs. Additionally since some of the modes share connections with other modes, for example in ⅞ mode and 6/8 mode C1 and C2 have the same connections (other than output and C3) and C3 is not used in 6/8 mode, the phases can be combined and a four-phase pump with two outputs would be created. It can be seen that the M=4 mode is available with any other mode, the M=2 mode is available with either M=1 or M=3, and that M=6 is available with M=7 or M=5, making any of these combinations available in four phases as well.

Converter 120 is used to efficiently power implantable medical device 5 as follows. Device 5 includes microcomputer circuit 114 and digital circuit 140 which are optimally powered at 1.2 volts, and analog circuit 150 that is optimally powered at 1.8 volts. Regulator 190 is connected between the V_{AOUT} output and analog circuit 150 and has a minimum dropout voltage of about 100 millivolts. The battery for this operation has an initial voltage of 3.5 volts.

TABLE III shows the values of M for various values of the battery voltage over its useful life:

TABLE III

| Battery | Digital supply (V_{DOUT}) | Analog supply (V_{AOUT}) |
|---|---|---|
| voltage (V_{IN}) | M setting | M setting |
| 3.5 to 3.2 | 3 | 5 |
| 3.5 to 3.05 | 4 | 5 |
| 3.05 to 2.55 | 4 | 6 |
| 2.55 to 2.4 | 4 | 7 |
| 2.4 to 2.2 | 5 | 8 |
| | | (running directly from |
| | | the battery) |
| 2.2 | 6 | 8 |
| to battery | | (running directly from |
| depletion | | the battery) |

Using converter 120 in conjunction with regulator 190 at the V_{AOUT} output provides an efficiency of approximately 87% over the battery's life. By comparison, the efficiency of an LDO regulator alone would be less than 60%. In addition the efficiency is very similar to the efficiency that could be obtained from a switching regulator, but converter 120 does not have the EMI noise/susceptibility problem and does not suffer from magnetic saturation.

A method of determining connections of the capacitors of an

$$\frac{M}{{2}^{N}}$$

charge pump converter during N+1 phases so that the voltages across the capacitors and the output voltage are all uniquely determined can be generalized in the following series of steps:

1. Express the desired input voltage to output voltage as a fraction, i.e. V_{IN}/V_{OUT}.

2. Determine M and N to generate an expression of

$$\frac{M}{{2}^{N}}$$

that has the desired accuracy, with M odd, which yields the lowest N (and thus the lowest number of capacitors needed to generate V_{OUT} to the accuracy desired).

3. Determine how the desired output can be generated by division of the nearest two of the next lower multiple of ½.

4. Repeat step 3 until all remaining powers are ½.

5. Generate a 1/2 ratio by connecting C1− to VSS in the first phase and C1+ to V_{IN} in the second phase.

6. Add or subtract each successive power of ½ by connection of the next capacitor to the unspecified connections of the nearest off multiple of the previous power of ½. If the next stage needs to be lower than the previous stage, the positive terminal is connected to the odd multiple phases.

7. Add a phase with the connection of the capacitor added in step 2 to the nearest even multiple of the previous power of ½, with the connection made in the opposite polarity on the connection to the odd multiple. If the nearest even multiple of the previous power of ½ has mode than one phase (i.e. 2/4 comes from ½, which has 2 phases) any of the phases may be selected to create this additional connection. Alternatively, all phases of the nearest even multiple may be selected, creating additional pump phases.

8. Repeat steps 2 and 3.

9. The output is connected to the unspecified node of the last capacitor for which connections were determined.

10. Re-arrange phases for convenience, such as to minimize the amount of switching or to accommodate a simultaneous output mode.

The following example illustrates how to implement the method outlined above for a particular case:

1. The desired V_{IN}/ V_{OUT} is determined to be a fraction close to 7/16.

2. M=7 and N=4 to generate a 7/16 ratio that has sufficient accuracy. M (=7) is odd, which yields the lowest N (=4) which determines the lowest number of capacitors (4) needed to generate V_{OUT} to the accuracy desired.

3. 7/16 is halfway between ⅜ and 4/8 (½).

4. ⅜ is halfway between ¼ and 2/4 (½). ¼ is halfway between ½ and 0/2 (0).

5. C1− is connected to V_{SS} in phase 1. C1+ is connected to V_{IN} in phase 2.

6-8. Generation of phases for ½:

C1− is connected to V_{SS} in phase 1.

C1+ is connected to V_{IN} in phase 2.

Generation of phases for ½:

C2+ is connected to C1+ in phase 1.

C2+ is connected to C1− in phase 2.

C2− is connected to Vₛₛ in phase 3.

Generation of phases for ⅜:

C3− is connected to C2− in phase 1.

C3− is connected to C2− in phase 2.

C3− is connected to C2+ in phase 3.

C3+ is connected to C1+ in phase 4 (C1− connected to V_{SS}).

Generation of phases for 7/16:

C4− is connected to C3+ in phase 1.

C4− is connected to C3+ in phase 2.

C4− is connected to C3+ in phase 3.

C4− is connected to C3− in phase 4.

C4+ is connected to C1+ in phase 5. (C1− connected to V_{SS}).

9. The output is connected to C4+ during phase 1, phase 2, phase 3, and phase 4. The output is connected to C4− during phase 5.

In the foregoing specification, the invention has been described with reference to specific embodiments. However, it may be appreciated that various modifications can be made without departing from the scope of the invention as set forth in the appended claims. For example in particular converters all M modes of operation may not be required and thus need not be supported. Furthermore only one output voltage may be required. A boosted voltage may be used as one of the modes. Also it is possible to further regulate the output voltage in multiple ways such as by using an output capacitor or a low dropout regulator. Accordingly, the specification and figures are to be regarded as illustrative rather than as restrictive, and all such modifications are intended to be included within the scope of the present invention.

What is claimed is:

1. A DC-to-DC converter suitable for use in an implantable medical device comprising:
N capacitors wherein n is a whole number greater than one; and
a controller coupled to each of said n capacitors and to an output voltage terminal for providing an output voltage thereat, and having N+1 phases wherein:
during a first phase of said N+1 phases said controller couples selected ones of said N capacitors to said output voltage terminal; and
during successive N phases of said N+1 phases said controller couples other selected ones of said N capacitors selectively between an input voltage terminal, said output voltage terminal, and a power supply voltage terminal such that voltages across each capacitor of said N capacitors and at said output voltage terminal assume respective uniquely determined values during all of said N+1 phases.

2. The DC-to-DC converter of claim 1 wherein during said first phase of said N+1 phases said controller couples all of said N capacitors in series between said output voltage terminal and said power supply voltage terminal.

3. The DC-to-DC converter of claim 2 wherein during said successive N phases of said N+1 phases said controller couples a first capacitor of said N capacitors between said input voltage terminal and said output voltage terminal.

4. The DC-to-DC converter of claim 3 wherein said controller couples an additional capacitor of said N capacitors besides said first capacitor between said input voltage terminal and said output voltage terminal during all remaining successive phases of said N+1 phases.

5. The DC-to-DC converter of claim 1 wherein said controller comprises a mode selector having an input terminal for receiving a feedback signal, and an output terminal for providing a mode signal based on said feedback signal.

6. The DC-to-DC converter of claim 5 further comprising an output capacitor having a first terminal coupled to said output voltage terminal, and a second terminal coupled to said power supply voltage terminal.

7. The DC-to-DC converter of claim 6 further comprising a regulator having a first terminal coupled to said output voltage terminal, and a second terminal for providing a regulated output voltage.

8. The DC-to-DC converter of claim 7 wherein said feedback signal is derived from said regulated output voltage.

9. The DC-to-DC converter of claim 5 wherein said controller further comprises:
a phase clock generator having an output for providing at least N+1 phase clock signals;
a switch control signal generator having a first input coupled to said mode selector for receiving a mode signal therefrom, a second input coupled to said phase clock generator for receiving said N+1 phase clock signals, and an output for providing a plurality of switch control signals; and
a switch network coupled to said input voltage terminal, said output voltage terminal, said power supply voltage terminal, and to each of said N capacitors, for switching first and second terminals of each of said N capacitors in response to said plurality of switch control signals.

10. The DC-to-DC converter of claim 1 wherein said controller comprises a single integrated circuit.

11. A DC-to-DC converter suitable for use in an implantable medical device comprising:
N capacitors, wherein N is a whole number greater than one; and
a controller coupled to each of said N capacitors and to an output voltage terminal for providing an output power supply voltage and having N+1 phases and operable in 2^{N} modes of operation wherein said controller switches said N capacitors during said N+1 phases such that in a mode M said controller provides a voltage at said output voltage terminal equal to M/2^{N} times a voltage at an input voltage terminal, wherein M is a whole number from 1 to 2^{N}.

12. The DC-to-DC converter of claim 11 wherein said controller comprises a mode selector having an input terminal for receiving a feedback signal, and an output terminal for providing a mode signal corresponding to M wherein said mode selector selects a value of M based on said feedback signal.

13. The DC-to-DC converter of claim 12 further comprising a regulator having a first terminal coupled to said output voltage terminal, and a second terminal for providing a regulated output voltage.

14. The DC-to-DC converter of claim 13 wherein said feedback signal is derived from said regulated output voltage.

15. The DC-to-DC converter of claim 12 wherein said mode selector is capable of providing said mode signal for all values of M between 1 and 2^{N}.

16. The DC-to-DC converter of claim 12 wherein said controller further comprises:
a phase clock generator having an output for providing at least N+1 phase clock signals;
a switch control signal generator having a first input coupled to said mode selector for receiving a mode signal therefrom, a second input coupled to said phase clock generator for receiving said N+1 phase clock signals, and an output terminal for providing a plurality of switch control signals; and
a switch network coupled to said input voltage terminal, said output voltage terminal, said power supply voltage terminal, and to each of said N capacitors, for switching first and second terminals of each of said N capacitors in response to said plurality of switch control signals.

17. The DC-to-DC converter of claim 11 wherein said controller comprises a single integrated circuit.

18. An implantable medical device comprising:
a sensor adapted to be coupled to living tissue;
a monitor circuit coupled to said sensor for processing inputs received from said sensor and powered with an output power supply voltage;
a power source; and
a DC-to-DC converter having an input coupled to said power source and an output coupled to said monitor circuit for providing said output power supply voltage thereto, comprising:
N capacitors, wherein N is a whole number greater than one; and
a controller coupled to each of said N capacitors and to an output voltage terminal for providing said output power supply voltage thereto, and having N+1 phases wherein:
during a first phase of said N+1 phases said controller couples selected ones of said N capacitors to said output voltage terminal;
during successive N phases of said N+1 phases said controller couples other selected ones of said N capacitors selectively between an input voltage terminal, said output voltage terminal, and a power supply voltage terminal, such that voltages across each capacitor of said N capacitors and at said output voltage terminal assume respective uniquely determined values during all of said N+1 phases.

19. The implantable medical device of claim 18 wherein said monitor circuit comprises a digital circuit and an analog circuit.

20. The implantable medical device of claim 19 wherein said DC-to-DC converter provides said output power supply voltage to said analog circuit and further has a second output for providing a second output power supply voltage to said digital circuit.

21. The implantable medical device of claim 20 wherein said digital circuit comprises a microcomputer circuit and a digital portion of an input/output circuit.

22. A method for converting an input voltage to an output voltage comprising the steps of:
providing N capacitors wherein N is a whole number greater than one;
generating at least N+1 phases;
coupling selected ones of said N capacitors to an output voltage terminal during a first phase; and
coupling other selected ones of said N capacitors selectively between an input voltage terminal, said output voltage terminal, and a power supply voltage terminal during each remaining phase such that voltages across each capacitor of said N capacitors and the output voltage assume respective uniquely determined values during all of said N+1 phases.

23. The method of claim 22 further comprising the step of coupling other selected ones of said N capacitors to a second output voltage terminal during at least another one of said N+1 phases to form a second output voltage.

24. The method of claim 22 wherein said step of coupling selected ones of said N flying capacitors in said first configuration comprises the step of coupling selected ones of said N capacitors in a serial configuration selectively between an input terminal, an output voltage terminal, and a power supply voltage terminal.

25. The method of claim 22 wherein said step of coupling other selected ones of said N capacitors during each remaining phase in respective configurations comprises the step of coupling other selected ones of said N capacitors during each remaining phase in respective serial configurations selectively between said input terminal, said output voltage terminal, and said power supply voltage terminal.

26. The method of claim 22 further comprising the steps of:
selecting a first mode of operation M wherein M is a whole number from 1 to 2^{N}; and
performing said steps of coupling said selected ones of said N capacitors in said first configuration during said first phase and coupling said other selected ones of said N capacitors during each remaining phase in respective configurations, such that said output power supply voltage assumes a value of M/2^{N}.

27. The method of claim 26 further comprising the step of performing said steps of selecting and performing for all values of M from 1 to 2^{N}.

28. The method of claim 26 further comprising the steps of:
receiving a feedback signal derived from the output voltage; and
changing said first mode of operation to a second mode of operation based on said feedback signal.
